(19) United States
(12) Patent Application Publication
(10) Pub. No.: US 2026/0038635 A1
(43) Pub. Date: Feb. 5, 2026

(54) ARTIFICIAL INTELLIGENCE-GUIDED MARKER ASSISTED SELECTION

(71) Applicant: PIONEER HI-BRED INTERNATIONAL, INC., JOHNSTON, IA (US)

(72) Inventors: JUSTIN P. GERKE, URBANDALE, IA (US); DAVID HABIER, JOHNSTON, IA (US); JHONATHAN PEDROSO RIGAL DOS SANTOS, JOHNSTON, IA (US); SIMON RENNY-BYFIELD, COLORADO SPRINGS, CO (US); ELI RODGERS-MELNICK, JOHNSTON, IA (US)

(73) Assignee: PIONEER HI-BRED INTERNATIONAL, INC., JOHNSTON, IA (US)

(21) Appl. No.: 18/996,439

(22) PCT Filed: Jul. 19, 2023
(86) PCT No.: PCT/US2023/070492

§ 371 Date: Jan. 17, 2025

Related U.S. Application Data
(60) Provisional application No. 63/368,987, filed on Jul. 21, 2022.

(51) Int. Cl. G16B 35/00; G16B 10/00; G16B 20/00; G16B 40/20

(52) U.S. Cl. CPC G16B 35/00; G16B 10/00; G16B 20/00; G16B 40/20

(57) ABSTRACT

The present disclosure provides improved breeding methods that allow for the selection of a member or members of a population having a desired phenotype using a limited number of genetic markers. In certain aspects, the methods for selecting members of the breeding program utilize machine learning models to predict phenotypes of a simulated progeny population.

### FIELD

This disclosure relates generally to the field of plant breeding, and more specifically, to the use of artificial intelligence and machine learning in plant breeding.

### BACKGROUND

One of the critical steps of plant breeding is the selection of progeny having a genotype that produces plants have the desired phenotypic characteristics. Current methods to select progeny having a preferred genotype are limited due to high costs and low efficiency.

For example, in maize breeding the genetic merit of recovered double-haploid embryos is not considered until the after most costly steps of the process. Directional selection guided by estimated Genomic Breeding Values (GEBVs) could potentially support transplanting only the best plants under a budget restriction. However, even moderate density genotyping required for genomic prediction (GP) of GEBVs is still financially unfeasible, due to the enormous numbers of generated embryos.

Marker-assisted selection (MAS), where individuals are genotyped at a few well characterized quantitative trait loci (QTLs), has been developed as a way to reduce genotyping costs associated with selection of breeding material. However, despite MAS being a plausible solution for reducing the number of genetic markers needed to predict a phenotype, it is still limited in that (i) many studies show a lower performance compared to GP; (ii) it requires generating experimental populations in the field; (iii) QTLs are population-specific and do not leverage the broader germplasm; and (iv) production of the QTL mapping populations is costly.

Accordingly, there is need to develop new methods for identifying markers associated with traits across a broad germplasm population and for selecting genotypes that produces plant having the desired phenotypic characteristics. This disclosure provides such methods.

### SUMMARY

Provided herein is a method for selecting a plurality of members for a breeding program comprising crossing at least two breeding partners in silico to create a simulated progeny population comprising a plurality of members, inputting representations of genotypic information from the simulated progeny population into a trained a machine learning model to generate phenotypic predictions for at least one trait of interest for one or more members of the plurality of members of the simulated progeny population, the machine learning model having been trained to predict the at least one trait of interest, identifying quantitative trait loci (QTL) associated with the at least one trait using the genotypic information and phenotypic profile of the plurality of members, and identifying at least one allele of one or more polymorphic markers within or linked to the identified QTL, wherein the markers are polymorphic within the population.

In certain embodiments, the method further comprises selecting a set of the one or more polymorphic markers that selectively identifies at least one member of the plurality of members having a desired genomic estimated breeding value (GEBV) for the at least one trait of interest.

In certain embodiments, the method further comprises generating a progeny population by crossing the at least two breeding partners and screening one or more members of the progeny population for the presence or absence of the polymorphic markers associated with the at least one trait of interest. In certain embodiments, progeny comprising a desired set of polymorphic markers for the at least one trait of interest are selected for further use in the breeding program.

### BRIEF DESCRIPTION OF THE DRAWINGS

The disclosure can be more fully understood from the following detailed description and the accompanying drawings, which form a part of this application.

FIG. 1 is a boxplot of experimental data providing the Pearson correlation coefficient (Pearson's r) from a comparison of the BayesABC based AI-MAS predictions vs traditional whole genome prediction (WGP) with the full marker set. The traits are: EARHT, ear height in inches; MST, kernel moisture percentage; TSTWT, kernel test weight; YIELD, yield in bushels per acre.

FIG. 2 is a boxplot of experimental data providing the Pearson correlation coefficient (Pearson's r) from a comparison of the BayesABC based AI-MAS predictions vs BLUP phenotypic values with the full marker set. The traits are: EARHT, ear height in inches; MST, kernel moisture percentage; TSTWT, kernel test weight; YIELD, yield in bushels per acre.

FIG. 3 provides a scatter plot showing AI-MAS predictions for F₂ filial lines vs mean value of “full” predictions for their derived F₄ filial lines (the trait of interest is yield). The plot shows data for a single family of 25 F₂s, and 50 derived F₄ for each F₂. The Pearson's correlation coefficient between neural net “full” predictions and AI-MAS prediction.

FIG. 4 is a graph providing the distributions of predictive yield values pre- and post-AI-MAS selection in a single exemplar family. The distribution of predicted phenotypic values for yield in an original population of simulated F₄ and the distribution of predicted phenotypic values for yield in an F₄ population derived from selected F₂ filial lines. A clear shift in the mean and median is evident where F₄ derived from selected F₂ filial lines tend to demonstrate improved phenotypic predictions.

FIG. 5 is a graph providing the distribution of within family correlation between AI-MAS predictions for yield vs “full” neural net predictions for yield. A total of 543 families are included. Each family consists of 25 F₂ filial lines and 50 F₄ filial lines from each F₂.

FIG. 6 is a boxplot showing the distribution of trait 8 values (calculation described in the text). Delta values show the degree of movement of the population mean post AI-MAS selection. In this case positive differences are favored and demonstrate that populations post-selection generally have more desirable phenotypes relative to the original unselected populations. The figure indicates that AI-MAS is predicted to perform well across several traits and the inbred heterotic groups of corn (Stiff-stalk and Non-stiff-stalk).

FIG. 7 is a boxplot showing the distribution of within family Pearson's r of BayesA based AI-MAS predictions vs whole genome prediction (WGP) values in soybean.

FIG. 8 is a boxplot showing the distribution of within family Pearson's r of ANN based AI-MAS predictions vs whole genome prediction (WGP) values in soybean.

### DETAILED DESCRIPTION

The present disclosure provides improved breeding methods that allow for the selection of a member or members of a population (e.g., members of a breeding program) having a desired phenotype using a limited number of genetic markers. The optimal choice of the genetic markers and their predicted effects on phenotype, for the methods provided herein, are generated from a machine learning model representation of the genetic variation and related phenotypes within the germplasm.

In certain embodiments, the method comprises inputting genotypic information from a population (e.g., simulated population) into a machine learning model to generate a predicted phenotypic profile for at least one trait of interest for the members of the population and identifying at least one allele of one or more polymorphic markers within or linked to the at least one trait. In certain embodiments, the method further comprises assigning an additive effect to the allele or alleles within or linked to the at least one trait.

Any population of interest may be used with the methods described herein. While the methods disclosed herein are exemplified and described primarily using plant populations, the methods are equally applicable to animal populations, for example, non-human animals, such as domesticated livestock, laboratory animals, companion animals, etc. The animal may be a poultry species, a porcine species, a bovine species, an ovine species, an equine species, or a companion animal, and the like. Accordingly, in some embodiments, the population is a population of plants or animals, for example, plant or animal populations for use in a breeding program. In some examples, the populations include populations of inbred plants, hybrid plants, microspores (also referred to herein as microspore embryos), haploid embryos, doubled haploid plants, (e.g., plants derived from microspores or haploid embryos), including but not limited to F1 or F2 doubled haploid plants, offspring or progeny thereof, including those from in silico crosses (e.g., simulated progeny population), or any combination of one or more of the foregoing. Any monocot or dicot plant may be used with the methods and compositions provided herein, including but not limited to a soybean, maize, sorghum, cotton, canola, sunflower, rice, wheat, sugarcane, alfalfa, tobacco, barley, cassava, peanuts, millet, oil palm, potatoes, rye, or sugar beet plant. In some embodiments, the genotypic data and/or phenotypic data is obtained from a population of soybean, maize, sorghum, cotton, canola, sunflower, rice, wheat, sugarcane, alfalfa, tobacco, barley, cassava, peanuts, millet, oil palm, potatoes, rye, or sugar beet plants. The population (e.g., simulated progeny population) of the methods described herein comprises a plurality (i.e., more than one) of members. The number of members may be determined based on the genome of the population (e.g., haploid, diploid, triploid, tetraploid) and/or the at least one trait of interest. In certain embodiments, the population comprises at least 5, 10, 50, 100, 150, 200, 250, 300, 350, 400, 500, 1000, 10000, 100,000 or 1,000,000 members and less than 5,000,000, 500,000, 5000, 1000, 500, 400, 350, 300, 250, 200, 150, or 100 members.

In certain embodiments of the methods described herein, the population is a progeny population produced by crossing two or more breeding partners, in which the genotype of the members is known. In certain embodiments, when the genotype of one or more of the breeding partners in unknown the method further comprises genotyping the breeding partner.

In certain embodiments, the population for use in the methods described herein is a population generated from an in silico simulation (e.g., simulated population). In certain embodiments, the population for use in the methods described herein is an in silico simulated progeny population produced by crossing at least 2 (e.g., 2, 3, 4, 5, 6, 7, 8, 9, 10 or more) breeding partners in silico. In certain embodiments, the simulated population (e.g., simulated progeny population) is a doubled haploid population, a haploid population, a microspore population, or a population of progeny from a bi-parental cross.

The genotypic profile (also referred to herein as genotype) of the members of the simulated populations described herein (e.g., simulated progeny population) may be simulated using any method known in the art. A “genomic profile” as used herein generally refers to a set of information about the entire genome of a given plant or group of plants (genome-wide), or it can encompass a specific subset of the genome of a given plant or group of plants, or any combination thereof in a given plant or group of plants. In certain embodiments, the genomic profile of the members of the simulated progeny population includes information regarding the presence or absence in the genome of a specific set of mutations, single nucleotide polymorphisms (SNPs), insertion of bases, deletion of bases, genotypic markers, other sequence information, or any combination thereof.

In certain embodiments, the genotypic profile of the members of the simulated progeny population is simulated using variational autoencoders (VAEs). For example, in certain embodiments, to predict the genotypes of the simulated progeny parental SNPs are imputed using VAEs trained for optimal reconstruction of the population, such as, for example, samples from a breeding program. Non-limiting examples of using VAEs trained for optimal reconstruction include, but are not limited to, those found in U.S. Pat. No. 11,174,522. In certain embodiments, the VAEs produce intermediate latent representations that could be decoded into imputed SNPs. The genotype of the members of the simulated progeny population can be simulated from the imputed SNPs. In certain embodiments, the genotypes of members of a simulated progeny population can be simulated using a statistical model of recombination. In certain embodiments, the statistical model of recombination is based on a Poisson process. For example, in certain embodiments to simulate SNPs based on a Poisson process recombination break points in the genetic map may be sampled from an exponential distribution with a rate parameter equal to one, and the sampled distance multiplied by 100 for conversion to centimorgans. In certain embodiments, the crossover interference rate can be sampled from a gamma distribution with shape and scale parameters equal to two. As would be understood by a person of ordinary skill in the art, the exact parameters to simulate the population using a statistical model of recombination based on the Poisson process may be adjusted based on the complexity of the genome of the population.

VAEs are hybrids of deep neural networks and probabilistic graphical models that enable construction of a compressed latent representation that is independent of the underlying data generation (e.g., genotyping platform) and serves as a basis of imputing characteristics of a desired data set (e.g., multiple germplasm characterization). The core of VAEs is rooted in Bayesian inference, which includes modeling of the underlying probability distribution of data, such that new data can be sampled from that distribution, which is independent of the dataset that resulted in the probability distribution. VAEs have a property that separates them from standard autoencoders that is suitable for generative modeling: the latent spaces that VAEs generate are, by nature of the framework, probability distributions, thereby allowing simpler random sampling and interpolation for desirable end-uses. VAEs accomplish this latent space representation by making its encoder not output an encoding vector of size n, rather, outputting two vectors of size n: a vector of means, u, and another vector of standard deviations, σ. Some of the basic notions for VAE include for example:
X: data that needs to be modeled, for example, genotypic data (such as SNPs, markers, haplotype, sequence information)
z: latent variable
P(X): probability distribution of the data, for example, genotypic data
P(z): probability distribution of latent variable (e.g., genotypic associations from the underlying genotypic data)
P(X|z): distribution of generating data given latent variable, e.g. prediction or imputation of the desired outcome based on the latent variable.

VAE is based on the principle that if there exists a hidden variable z, which generates an observation or an outcome x, then one of the objectives is to model the data, i.e., to find P(X). However, one can observe x, but the characteristics of z need to be inferred. Thus, p(z|x) needs to be computed.

$$p ( z \vert x ) = p ( x \vert z ) p ( z ) / p ( x )$$

However, computing p(x) is based on probability theory, in relation to z. This function can be expressed as follows:

$$p ( x ) = \int p ( x \vert z ) p ( z ) dz$$

While the p(x) function is an intractable distribution, variational inference is used to optimize the joint distribution of x and z. The function p(z|x) is approximated by another distribution q(z|x), which is defined such that it is a tractable distribution. The parameters of q(z|x) are defined such that they are highly similar to p(z|x) and therefore, it can be used to perform approximate inference of the intractable distribution. KL divergence is a measure of difference between two probability distributions. Therefore, if the goal is to minimize the KL divergence between the two distributions, this minimization function is expressed as:

$$min KL ( q ( z \vert x ) ‖ p ( z \vert x ) )$$

This expression is minimized by maximizing the following:

$$E q ( z \vert x ) log p ( x \vert z ) - K L ( q ( z \vert x ) ‖ p ( z ) )$$

Reconstruction likelihood is represented by the first part, and the second term penalizes departure of probability mass in q from the prior distribution, p. q is used to infer hidden variables (latent representation) and this is built into a neural network architecture where the encoder model learns the mapping relation from x to z and the decoder model learns the mapping from z back to x. Therefore, the neural network for this function includes two terms-one that penalizes reconstruction error or maximizes the reconstruction likelihood and the other that encourages the learned distribution q(z|x) to be highly similar to the true prior distribution p(z), which is assumed to follow a unit Gaussian distribution, for each dimension j of the latent space. This is represented by:

$$ℒ ( x , \hat{x} ) + {\sum }_{j} KL ( {q}_{j} ( z \vert x ) ‖ p ( z ) )$$

It should be appreciated that the variational autoencoder is one of several techniques that may be used for producing compressed latent representations of raw samples, for example, genotypic association data. Like other autoencoders, the variational autoencoder places a reduced dimensionality bottleneck layer between an encoder and a decoder neural network. Optimizing the neural network weights relative to the reconstruction error then produces separation of the samples within the latent space. However, unlike generative adversarial networks (GAN), the encoder neural network's outputs are parameterized univariate Gaussian distributions with standard N(0,1) priors. Thus, unlike other autoencoders, which tend to memorize inputs and place them in arbitrarily small locations within the latent space, the variational autoencoder produces a smooth, continuous latent space in which semantically-similar samples tend to be geometrically close—e.g., haplotypes that co-segregate to provide a certain phenotype.

For example, in the context of genomic characterization, a smooth spatial organization of the latent space captures varying levels of ancestral relationships that are present within a dataset. Genomic variation within a population such as a plant breeding program may be characterized by a variety of methods. For example, genotypes are characterized with a common platform that interrogates localized variants such as single nucleotide polymorphisms (SNPs) and/or insertions/deletions (indels). Due to the ancestral recombination and demographic history of the population, these variants tend to co-segregate within linked segments (haplotypes). Further, single genotypes may then be further characterized by the set of haplotypes they contain. For example, as described further below, VAEs may be used to compress the information contained within a given set of production markers to a common, marker-invariant, latent space capable of capturing these co-segregation patterns genome-wide.

Any suitable machine learning model may be used in the in the methods and systems described herein. Types of models include without limitation statistical models, such as probability models, regression models, and those involving deep learning, such as supervised and unsupervised models, or combinations thereof. In certain embodiments, the machine learning model is a classification model, a regression model, a clustering model, a dimensionality reduction model, retrospective index model, a distribution model, for example, a multivariate or univariate Gaussian distribution model, or a deep learning model. In certain embodiments, the deep learning model is part of an ensemble model. In certain embodiments, the deep learning model is an ensemble model comprising two or more models. In certain embodiments, the deep learning model is a supervised learning model. The supervised learning model may be a classification or regression model. The machine learning models include support vector machines, neural networks, such as SVM-DA (Support Vector machines) or ANN (Artificial Neural Networks), or deep learning algorithms and the like.

In certain embodiments, the machine learning model for use in the methods described herein is an artificial neural network (ANN). ANNs are configured to synthesize or learn from a plurality of inputs to produce an output—for example, one or more inputs to disease resistance information can be modeled using machine learning approaches involving Bayesian algorithms. One or more variables in the algorithms can have weights that are applied to each equation and optimized as the neural network is trained. Based on the amount of training information, the deep learning models or networks get better at producing more helpful outputs. In certain embodiments, the ANN includes a plurality of input factors that may be used to train predicted phenotypic information. These factors include, but are not limited to, QTLs, SNPs, haplotypes, yield, and other historical agronomic or breeding phenotypic components.

Training data generally refers to datasets that are used to train specific deep learning networks, such as for example, artificial neural network. Each dataset may correspond to set of actual trait values (e.g., yield value) and the underlying genotype for a plant. Yield values for example, represent grain yield. Other trait values such as, for example, the phenotypic traits of interest described herein can be utilized to train the machine learning models (e.g., ANN) described herein. Training datasets can be used with various types of machine learning algorithms such as supervised learning, unsupervised learning, semi-supervised learning, and reinforcement learning. Neural network algorithm is an example of supervised learning—where a special purpose computer or a computing system is provided with training data containing the input/predictors along with the correct output. From the training data the computer/algorithm should be able to learn the patterns. Supervised learning algorithms model associations and dependencies between the target prediction output and the input features such that the output values for new data based on those previous associations that the network learned from. Training datasets can include measured data, simulated data, or a combination thereof.

In certain embodiments, the ANN for use in the methods described herein is trained for a set of traits of interest (e.g., yield, plant height, seed moisture, test weight, growing degree days for silking, ear height, brittle snap, early root lodging, late root lodging, or resistance to northern corn leaf blight) In certain embodiments, the ANN is trained separately for each trait of interest. In certain embodiments, the ANN is trained using a hold-one-year-out strategy. In certain embodiments, separate ANNs for each heterotic group are trained simultaneously using, for example, a variant of stochastic gradient descent (e.g., predicted phenotype is modeled as the sum of the separate neural network outputs). In certain embodiments, inputs to the ANNs described herein include both latent representations of the genotypes and learned vectors for each mega-environment in the dataset. In certain embodiments, the ANN is trained using an objective function. In certain embodiments, the objective function accounts for contrasts within environment, which, for example, aids in distinguishing within-family effects and differing environmental variances. In certain embodiments, the ANN genotype representations comprise representations sampled from the genotypic latent space. In certain embodiments, a model of main environmental effects is fit prior to training of the genomic predictor.

In certain embodiments, the machine learning model for use in the methods described herein is a Bayesian genomic prediction model (e.g., BayesA, BayesB, or BayesC). In certain embodiments, the training dataset for the Bayesian genomic prediction model comprises phenotypes for the trait or traits of interest and marker genotype scores from a plurality of material representative of the germplasm of interest. For example, when the simulated progeny population is a maize double haploid, the representative material is historical maize populations. In certain embodiments, the training dataset is used to estimate marker effects for the trait of interest in the population (e.g., simulated progeny population) using the prediction model BayesA, BayesB, and/or BayesC.

As used herein, a “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern blot analysis, RT-PCR, microarray gene expression assays, or reporter gene expression systems, or by agricultural observations such as stress tolerance, yield, or pathogen tolerance.

As used herein a “trait of interest” refers to any phenotypic trait used for the selection of members of a breeding population. Exemplary traits of interest include, but are not limited to, yield, disease resistance (e.g., phytophthora tolerance, Fusarium solani tolerance, stem rot tolerance, Asian soybean rust resistance, frogeye leaf spot tolerance, anthracnose stalk rot resistance, northern corn leaf blight resistance, corn lethal necrosis resistance, common smut resistance, common rust resistance, diplodia mold resistance, diplodia stalk rot resistance, eyespot resistance, fusarium ear rot resistance, gibberella ear rot resistance, gibberella stalk rot resistance, gray leaf spot resistance, goss' wilt resistance, brown stem rot tolerance, soybean cyst nematode resistance), agronomic traits (e.g., brittle snap, early root lodging, late root lodging, relative maturity, growing degree days for silking, drydown, flowering time, pod shatter), abiotic traits (e.g., drought tolerance, iron deficiency tolerance, chloride tolerance, temperature, nitrogen use efficiency), seed and/or kernel composition (e.g., moisture, test weight, protein percentage, oil percentage, meal type, oleic acid percentage, linoleic acid percentage, linolenic acid percentage), herbicide tolerance, insect resistance, fertility (e.g., pollen shed), silage yield, and morphological traits (e.g., ear height, plant height).

In certain embodiments of the methods provided herein, after generating the predicted phenotype of the members of the population the method further comprises identifying quantitative trait loci (QTLs) associated with the at least one trait of interest. A QTL, as used herein, refers to a region of DNA which is associated with a particular phenotype (e.g., trait of interest). In certain embodiments, quantitative trait loci (QTLs) are identified by QTL mapping using the genotypic information of the population and the corresponding predicted phenotype to identify QTLs associated with the trait or traits of interest. The method of QTL mapping is not particularly limited and may be any method known in the art to identify QTLs, such methods include, but are not limited to, analysis of variance, interval mapping, standard interval mapping, simple interval mapping, composite interval mapping (CIM), and family-pedigree based mapping. In certain embodiments, the QTL mapping is performed using CIM. The CIM method estimates one QTL effect at a time using its flanking markers, with markers from other regions as covariates in a linear regression model leveraging their additive effects to control linkage disequilibrium, increasing detection power and resolution.

In certain embodiments, after QTLs associated with the at least one trait are identified the method further comprises identifying at least one allele of one or more polymorphic molecular markers within or linked to the identified QTL. In certain embodiments, the molecular marker is a polymorphic molecular marker. In certain embodiments, the QTL comprises a single polymorphic marker. In certain embodiments, the QTL comprises a plurality of polymorphic markers.

The identified QTLs and polymorphic markers within or linked to the QTLs can vary in the degree of association with the phenotype. In certain embodiments, a QTL or polymorphic markers within or linked to the QTL for a trait is considered significant when the logarithm of odds (LOD) is greater than or equal to 1, 1.5, 2, 2.5, 3, 3.5, 4, 4.5, or 5. In certain embodiments, the QTL is considered significant when the LOD is greater than or equal to 2.

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in QTL mapping. In this case, the LOD score's size is dependent on the closeness of the marker locus to the locus affecting the quantitative trait, as well as the size of the quantitative trait effect.

As used herein a “polymorphic molecular marker”, “polymorphic marker” or the like refers to a molecular marker that is different in one sequence when compared to a related sequence when the two nucleic acids are aligned for maximal correspondence. As used herein, “marker”, “molecular marker”, “marker locus” or the like refers to a nucleic acid or amino acid sequence that is sufficiently unique to characterize a specific locus on the genome. Any detectible polymorphic marker can be used as a marker so long as it is inherited differentially and exhibits non-random association with a phenotypic trait of interest. Non-limiting examples of markers for use in the methods described herein include, but are not limited to, restriction fragment length polymorphisms (RFLPs), single sequence repeats (SSRs), target region amplification polymorphisms (TRAPs), randomly amplified polymorphic DNAs (RAPDs), variable number tandem repeat (VNTR), insertions/deletions (INDELs), amplified fragment length polymorphisms (AFLPs), and single nucleotide polymorphisms (SNPs). In certain embodiments of the methods described herein, the polymorphic markers are SNPs.

As used herein “additive effect” refers to the contribution of an allele of a polymorphic marker on the phenotype (e.g., trait of interest).

In certain embodiments, the additive effect is measured by the additive contrast between groups of members sharing different parental alleles from the same loci. Genomic estimated breeding values (GEBVs) are calculated for one or more of the identified QTLs associated with the trait. In certain embodiments, the GEBVs are calculated for QTLs having an LOD score greater than or equal to 2. Methods to calculate GEBVs are known in the art. In certain embodiments, the GEBV is calculated based on the sum of additive effects from polymorphic markers (e.g., SNPs) with the strongest additive effect within each detected QTL.

As used herein, “genomic estimated breeding values” (GEBVs) refer to a measurable degree to which one or more, polymorphic markers, haplotypes and/or genotypes heritably affect the expression of a phenotype associated with a trait.

In certain embodiments, the method further comprises ranking the additive effects of the identified polymorphic markers within or linked to QTLs associated with the trait of interest. In certain embodiments, the alleles are ranked based on their LOD score in which often a higher score corresponds to a greater additive effect.

By calculating the GEBVs, the polymorphic markers associated with the trait of interest are identified such that a limited set of markers can be used to identify members of a breeding population having the desired phenotype (e.g., trait of interest). Accordingly, in certain embodiments, the method comprises determining the least number of polymorphic markers, within or linked to the identified QTLs, that can predict the phenotype. As should be understood by a person of ordinary skill in the art the absolute number of polymorphic markers needed to identify a phenotype will vary based on the trait (e.g., continuous trait vs. discrete trait). In certain embodiments, the number of markers needed to predict the phenotype is less than the number of markers needed to predict the phenotype using genome prediction. In certain embodiments, the number of markers needed to predict the phenotype is at least 0.001%, 0.01%, 0.1%, 1%, 5%, 10%, 15%, 20%, 25%, 30%, 35%, 40%, 45%, 50%, 55%, 60%, 65%, 70%, 75%, 80%, 85%, 90%, or 95% fewer than the number of markers needed to predict the phenotype using genome prediction.

As used herein “genome prediction”, “whole genome prediction” and the like refers to prediction methods in which polymorphic markers densely distributed over the genome are used regardless of their association with the trait of interest. Examples of whole genome prediction can be found in, for example, Meuwissen et al. (Genetics, 157 (4): 1819-1829 (2001)) and de los Campos (Genetics, 193 (2): 327-345 (2013)). The GEBVs are often calculated but not limited to the sum of all additive effects estimated with a genomic prediction model.

In certain embodiments of the methods described herein, the method further comprises crossing the at least two breeding partners used to create the simulated progeny population to generate a progeny population and selecting progeny from the generated progeny population comprising the identified markers, thereby selecting progeny having the desired trait of interest. In certain embodiments, the method comprises detecting in the progeny population the polymorphic markers (e.g., markers within or linked to the identified QTL). The method for detecting is not particularly limited and may be any method known in the art to identify a polymorphic marker such as DNA sequencing or RT-PCR. In certain embodiments, once progeny having the trait of interest are selected, they are advanced in the breeding program to, for example, produce a commercial product or be used as a breeding partner. In certain embodiments, the markers are associated with a less desirable trait such that progeny comprising the identified polymorphic markers are removed from the breeding program. In certain embodiments, selected progeny having the trait of interest (e.g., progeny comprising the at least one allele of one or more polymorphic markers within or linked to the identified QTL) are self-pollinated and/or crossed to produce a population of plants having the trait of interest. For example, in certain embodiments, the method comprises crossing the at least two breeding partners to produce a progeny population, detecting in the nucleic acid of a plant or embryo of the progeny population the at least one allele of one or more polymorphic markers within or linked to the identified QTL, selecting a plant or embryo comprising the at least one allele. In certain embodiments, the method further comprises crossing the selected plant or a plant generated from the embryo with a second plant to produce a population comprising the at least one allele of one or more polymorphic markers, and collecting the seeds produced thereby.

In certain embodiments, the progeny population is a microspore embryo population or a haploid population (e.g., population produced by crossing a plant with a haploid inducer line). In certain embodiments, the methods described herein further comprise crossing the at least two breeding partners to produce a progeny microspore population or haploid embryo population, detecting in the microspore population or haploid embryo population the at least one allele of one or more polymorphic markers within or linked to the identified QTL, selecting a microspore or haploid embryo comprising the at least one allele, and generating a double haploid plant from the selected microspore or haploid embryo. Methods to produce double haploid plants from microspores are known in the art and include, but is not limited to, treatment with a chromosome doubling agent.

In certain embodiments, the steps of using the machine learning model to predict at least one trait of interest, identifying polymorphic markers and/or QTLs associated with the trait are repeated in the simulated progeny population for a different trait of interest, thereby identifying polymorphic markers associated with at least one additional trait of interest. As would be understood by a person of ordinary skill this process could be repeated any number of times based on the traits of interest.

In certain embodiments of the methods described herein, AI-MAS GEBVs can be generated for a trait of interest, individuals ranked and 1%, 5%, 10%, 15%, 20%, 25%, 30%, 35%, 40%, 45%, 50%, 55%, 60%, 65%, 70%, 75%, 80%, 85%, 90% and 95% of individuals selected from the population, with the remainder of the population being discarded. The smaller population of selected individuals can then be subjected to standard whole genome prediction techniques to generate genome-wide predictions (WGP GEBVs) for the target trait and/or any other trait of interest. Individuals can be ranked according to their WGP GEBVs, and final advancement decisions can be made on those WGP GEBVs.

In certain embodiments, the progeny from the breeding cross are selected for the multiple traits using the polymorphic markers identified for each trait. In certain embodiments, the method comprises weighting the traits and identifying markers that predict plants having the desired combination of traits. In certain embodiments, the method comprises (i) generating selection indexes among the set of traits, (ii) ranking progeny across two or more traits, (iii) calculating a weighted Czekanowski Coefficient, repeating steps (i)-(iii) 10 or more times (e.g., 10, 50, 100, 500, 1000, or 5000 or more).

The following are examples of specific embodiments of some aspects of the invention. The examples are offered for illustrative purposes only and are not intended to limit the scope of the invention in any way.

### Example 1

This example demonstrates the in silico generation of a simulated double haploid progeny population from a bi-parental cross.

To generate a simulated double haploid progeny population an in silico simulation of a bi-parental cross based on a statistical model of recombination was performed. Briefly, parental SNPs were imputed using variational autoencoders trained for optimal reconstruction of Corteva maize genetics, as previously described (U.S. Pat. No. 11,174,522). These autoencoders produced intermediate latent representations that could be decoded into imputed SNPs or phenotypes.

Using the imputed SNPs from parental inbred lines in 629 real-world maize historical biparental populations (variable family size), double-haploid (DH) populations were simulated with 23224 SNP markers under a population size of 200. For simulating the SNPs based on a Poisson process, recombination breakpoints in the genetic map were sampled from an exponential distribution with a rate parameter equal to one, and the sampled distance multiplied by 100 for conversion to centimorgans. In the same stochastic process, the crossover interference rate was sampled from a gamma distribution with shape and scale parameters equal to two.

The resulting simulated progeny population comprised of 200 genetically distinct double-haploid lines.

### Example 2

This example demonstrates the generation of an artificial neural network (ANN) machine learning model to predict a phenotype of the simulated progeny population.

In order to predict the phenotypes of the simulated progeny populations, machine learning models were developed. In one instance an artificial neural network (ANN) was trained for the traits: yield, plant height, seed moisture, test weight, growing degree days for silking, ear height, brittle snap, early root lodging, late root lodging, and northern corn leaf blight. Briefly, dense deep feed-forward neural networks were trained separately for each phenotype, using a hold-one-year-out strategy. Any hybrid genotype present within the held-out year was also held out of training within the set of training years. Separate neural networks for each heterotic group were trained simultaneously using a variant of stochastic gradient descent, wherein the predicted phenotype was modeled as the sum of the separate neural network outputs. Inputs to these neural networks included both the latent representations of the genotypes and learned vectors for each mega-environment in the dataset. By providing the neural networks with both genotypes and environments, the neural networks learned to predict general combining abilities (GCA) specific the regions of interest. Following training for 250 epochs, these neural nets could be used for prediction and—through in silico QTL mapping of simulated populations-inference of genetic effects.

All models were conditioned on evaluation in a given mega-environment, and each year between 2015-2019 was held-out of the training process to ensure any predicted phenotypes from the held-out year would reflect generalized performance.

For the same populations, the GCA was predicted from the pre-trained ANNs. In particular, the predicted phenotypes of a given progeny were obtained by feeding its genetic latent representations from the SNPs of the simulated progeny into the ANN, which was not trained with any data from the empirical progeny. This returned the predicted phenotype (predicted GCA) for the members of the simulated progeny population (e.g., in silico DHs) for the given mega-environment and trait of interest.

### Example 3

This example demonstrates the generation of a Bayesian genomic prediction machine learning model to predict a phenotype of the simulated progeny population.

In Example 2 the predicted phenotypes of the simulated progeny population were predicted with ANNs and markers (QTL) were identified with composite interval mapping (CIM) using those simulated genotypes and predicted phenotypes. In this example, an analogous process of phenotype prediction and marker identification and prediction is performed in which the ANNs and CIM are replaced with various Bayesian genomic prediction models. Each step of the following example has an exact analogous step in the original description of AI-MAS. For a given family the basic outline for performing AI-MAS using BayesABC is as follows:
[1] A training datasets is provided that contains phenotypes (BLUPs) for the trait of interest and marker genotype scores from a plurality of material targeting the germplasm of interest.
[2] The training dataset is used to estimate marker effects for the trait of interest using the Bayesian genomic prediction models BayesA, BayesB or BayesC.
[3] Using the previously described genetic simulation algorithms (or any other genetic simulation algorithm that utilized recombination to produce new genetic combinations) marker genotype scores for the full set of markers are generated for each of a plurality of simulants from the family of interest.
[4] The simulated marker genotype scores (from [3]) and the estimated marker effects (from [2]) are used to calculate a simulated true breeding value for each simulant from the family of interest.
[5] The simulated true breeding values of the family of interest are used to identify a candidate set of AI-MAS markers using a family-specific Genome-Wide Association Study (GWAS).
[6] A forward-stepwise Least Squares approach is used to both identify the final set of AI-MAS markers and estimate their effects for the family of interest. These markers explain the most variation of the simulated true breeding values of the simulants from the family of interest (from [4]).
[7] Genotype scores of the AI-MAS markers for real-life selection candidates from the family of interest and the estimated effects of the AI-MAS markers are used to calculate AI-MAS Genomic Estimated Breeding Values (AI-MAS-GEBVs) for the real-life selection candidates.

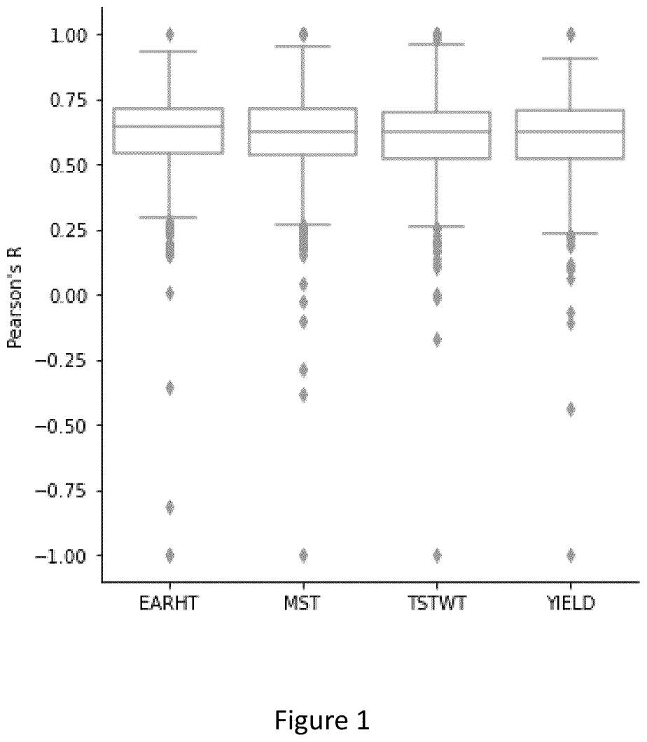

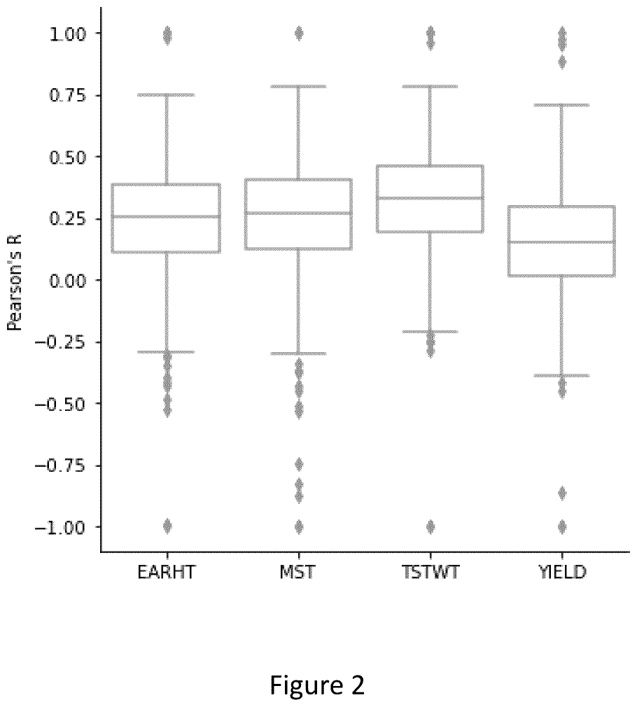

Bayesian genomic prediction-based AI-MAS prediction was validated using 762 unique families comprising a diverse set of maize germplasm from North America and Europe. For each family the within family Pearson's correlation coefficient (Pearson's R) was calculated between AI-MAS predictions and either [1] whole genome prediction (WGP) using the whole marker set (FIG. 1) or [2] phenotypic BLUPs (FIG. 2).

Comparing full WGP to AI-MAS predictions for the 762 families reveals a strong positive correlation between AI-MAS scores and their corresponding full predictions. The mean Pearson's R was between 0.60-0.62 depending on the trait, whilst the median Pearson's R was between 0.62-0.65. When comparing AI-MAS predictions to phenotypic BLUPs the mean Pearson's R was between 0.15-0.32, whereas the median Pearson's R was between 0.15-0.33.

Accordingly, this example demonstrates the use of a Bayesian genomic prediction machine learning model in the AI-MAS method.

### Example 4

This example demonstrates the application of artificial intelligence-guided marker-assisted selection (AI-MAS) with a trait of interest in a simulated double haploid progeny population.

Once the SNP markers and predicted phenotypes were available for the simulated progeny population, compositive interval mapping (CIM) analyses (Zeng, 1994) were performed to identify quantitative trait loci (QTL) underlying the phenotypes across the traits of the corresponding ANNs. Briefly, the CIM method estimates one QTL effect at a time using its flanking markers, with markers from other regions as covariates in a linear regression model leveraging their additive effects to control linkage disequilibrium, increasing detection power and resolution (Zeng, 1994).

For each significant QTL in each trait (logarithm of the odds (LOD)>2), genomic estimated breeding values (GEBVs) were calculated based on the additive contrast of the SNP alleles with the strongest absolute additive effect within each detected QTL (Collard et al., Phil. Trans. R. Soc. B 363:557-572 (2007)), setting the number of markers equal to the number of detected QTLs.

To assess the accuracy (Pearson correlation) of the predicted phenotype and corresponding QTL mapping a comparison to empirical data was performed. In particular, the simulated populations all had empirical phenotypic data available, allowing evaluation of prediction accuracy based on AI-MAS-derived GEBVs. These GEBV predictions were validated leveraging historical data from 1944 (test weight), 2344 (moisture), 2056 (ear height), 2376 (yield) overlapping set of single-cross maize hybrids across traits derived from 629 populations generated from 2015 to 2018, covering 7 mega-environments over agricultural lands from the United States of America (USA) and Europe. The calculation of the predicted GEBVs of the hybrids were based on the sum of additive marker effects from the hybrid parental double-haploid line derived from the biparental cross-tester inbred line additive effects were ignored in GEBV calculation.

On average, 8 markers (min-1, max-26) were used for calculating the GEBVs across scenarios. On average, the AI-MAS procedure showed a Pearson correlation value of (mean=0.36, std=0.20) to the mean phenotype calculated using BLUP methodology as ground truth (Piepho et al. 2008), compared to value of (mean=0.43, std=0.21) to the production standard linear model using genomic prediction (GP). Stratified for each trait over families, prediction accuracies for yield (AI-MAS: mean=0.26, std x=0.20; GP: mean=0.27, std=0.19), moisture (AI-MAS: mean-0.39, std-0.19; GP: mean-0.50, std-0.17), ear height (AI-MAS: mean-0.37, std=0.18; GP: mean=0.42, std=0.19), and test weight (AI-MAS: mean=0.43, std=0.18; GP: mean=0.57, std=0.17) were observed.

Thus, this example shows the ability to predict SNP markers and phenotypes of the simulated population using machine learning allowing for obtaining data for mapping QTL loci and selecting a few SNP markers predictive of performance in single-trait selection scenarios with DH populations.

### Example 5

This example demonstrates the application of AI-MAS with a trait of interest in a simulated single seed descent (SSD) progeny population.

Once the predicted phenotypes and SNP markers pairs were available for the simulated progeny population-DH progenies for QTL detection, QTL mapping was performed as described in Example 4; however, for the SSD populations (prediction target) optimal markers segregating as heterozygous indicated by the optimization analysis with segregation in the progeny were ignored when calculating the GEBVs of the candidates' progenies for selection. The remaining steps of methodology were the same for single traits.

With historical SSDs populations, data from 446 genetic elements from 21 biparental populations were used for validation. A density of 5 markers on average were used for building the GEBVs across scenarios. The AI-MAS procedure showed a Pearson correlation value of (mean=0.32, std=0.27) between the adjusted mean of phenotypes and predictions across evaluated conditions, compared to (mean-0.59, std-0.18) from the standard WGP approach. Stratified for each trait over populations, prediction accuracies of yield (AI-MAS: mean=0.24, std=0.30; GP: mean=0.49, std=0.15), moisture (AI-MAS: mean=0.25, std=0.25; GP: mean=0.59, std=0.21), ear height (AI-MAS: mean=0.34, std=0.28; GP: mean=0.55, std=0.17), and test weight (AI-MAS: mean=0.45, std=0.19; GP: mean=0.68, std=0.13) were observed.

Thus, this example shows the ability to predict SNP markers and phenotypes of the simulated SSD population using machine learning allows obtaining data for mapping QTL loci and selecting few SNP markers predictive of performance.

### Example 5

This example demonstrates a method for the identification of QTLs for the simultaneous selection of multiple traits

The AI-MAS procedure was extended for guiding the simultaneous selection of multiple traits (phenotypes) within a DH (evaluated with historical data) by leveraging the GEBVs derived from single-trait predictions and augmented with stochastic optimization to choose a superior subpopulation across traits. In general, we took the approach of finding a weighting of traits that would maximize the correspondence between selection on a combination of traits using the true breeding values and the realized selection under the information obtained from the AI-MAS markers.

In one instance of AI-MAS optimized for multiple traits, the steps comprise:
1. Stochastic optimization step
   1.1 Population simulation
   (i) Simulate markers and generate AI-MAS GEBV predictions under a fixed population size (example 1);
   1.2 Multi-trait superior subpopulation search
   (ii) Create selection indexes with all possible dual trait permutations (target, beneficial) among the target set of traits (study case: yield, moisture, ear height);
   (iii) Sample a selection intensity for a target trait (alpha), and for the beneficial trait (beta), such that the value for beta is greater than lambda and both are below 0.5
   (iv) Rank progenies across all traits, and create pools based on the intersection of superior progeny between target/beneficial trait combinations, Given the index calculated with alpha, beta, the intersection between the top ranks from each trait pair is appended to the target trait pool;
   (v) Sample one target trait pool (proportional to selection index) and sample one progeny within the target pool from a categorical uniform, and keep sampling until reaching a desired superior progeny size proportional to a joint intensity of selection and the fixed population size;
   1.3 Calculate fitness metric
   (vi) Calculate Weighted Czekanowski Coefficient (CC) of the superior progeny against the rank of true response (predicted GCA from NN+noise based on h2=0.5)-calculated marginally within traits and weight it by the selection index.
   1.4 Replicate process
   (vii) Repeat step (i)-(vi) 1000 times, and pick alpha and beta that maximizes the weighted CC;
2. Prediction step
   (viii) Replicate the process with AI-MAS GEBV predictions (real world), fixing the optimized alpha/beta hyperparameters;
   (ix) Get the final superior progenies for selection.

The procedure was validated using 87 historical populations evaluated from 2015 to 2018 in 8 mega-environments over the USA and Europe.

Validation metrics measure the ability to jointly select the traits: yield, moisture, and ear height. The following metrics were calculated:
1. Weighted CC for the top 25% AI-MAS-predicted superior progeny, and BLUP rank-oriented superior progeny (Weighted CC-BLUP);
2. Weighted CC for the top 25% AI-MAS-predicted superior progeny, and randomly sampled progeny (Weighted CC-RS)-averaged over 10 replicates.

The weighted CC-BLUP checks the agreement from superior progenies indicated by AI-MAS against those shown by E-BLUP (ground truth). Weighted CC-RS measures the same but with a random sample of progenies instead of the ground truth. The relative magnitudes of the Weighted CC-BLUP and the Weighted CC-RS denote the expected increase in genetic gain across traits using AI-MAS multi-trait optimization.

Using a set of populations (population number=86, mean population size=107, std population size=29) with historical data for multiple-traits, AI-MAS multi-trait optimization was found to be associated with increased genetic gain. It was obtained Weighted CC-BLUP (mean=0.27, std=0.12) 17.39% larger in average than the Weighted CC-RS (mean=0.23, std=0.11). With the CC calculated marginally within each trait-CC-RS averaged over 10 replicates, it was noticed for ear height CC-BLUP (mean=0.28, std=0.14) 28.00% larger than CC-RS (mean=0.23, std=0.12), for moisture CC-BLUP (mean=0.29, std=0.14) 29.00% larger than CC-RS (mean=0.24, std=0.12), and for yield CC-BLUP (mean=0.26, std=0.13) 26% larger than CC-RS (mean=0.23, std=0.13). Improvements are expected with larger population sizes displaying more segregants for multi-trait selection.

An analysis of the simultaneous selection of multiple traits in the SSD population using the same method as described above is being evaluated and validated.

Thus, this example shows the ability to use the machine learning model to obtain data for mapping QTL loci and selecting a few SNP markers predictive of multiple traits.

### Example 6

This example demonstrates the value of AI-MAS as compared to whole genome prediction.

The potential of AI-MAS to drive genetic gain varies by crop, breeding program, trait, geography, and family. One approach to assess the potential value of AI-MAS for a given family involves in-silico simulation of genotypes for filial lines produced by crosses between the two parents. For example, the correlation between AI-MAS phenotypic predictions for a given trait (produced with a handful of QTL markers) and predictions generated with a full complement of SNP markers (a “full” prediction) allows the assessment of the potential predictive power of the small set of AI-MAS QTL relative to full predictions. In this example, the potential of AI-MAS using 543 corn families from North America and Europe, and five key agronomic traits was assessed.

For each family, the standard AI-MAS approach is used to simulate a full set of marker genotypes for 200 doubled haploids (DHs), as in Example 1. Neural nets, trained to predict phenotypic values in specific breeding programs and years, were used to generate predicted phenotypic profiles for the simulated DHs using the full marker set (as in Example 3). Each trait was predicted separately but using the same set of markers. Following phenotypic prediction, the simulated genotypic values of the DHs, coupled with their predicted phenotypes, were combined with classical methods to identify a limited number of QTL for the trait of interest, as described above in Example 4. These QTL, and their associated additive effects, are used to predict the phenotypic value of real or simulated lines, provided marker scores were available at QTL. The number of SNP markers used in genotyping, simulations, and “full” predictions is arbitrary and can range from several hundred to potentially millions of markers. In contrast, AI-MAS predictions are based on a handful of SNP markers.

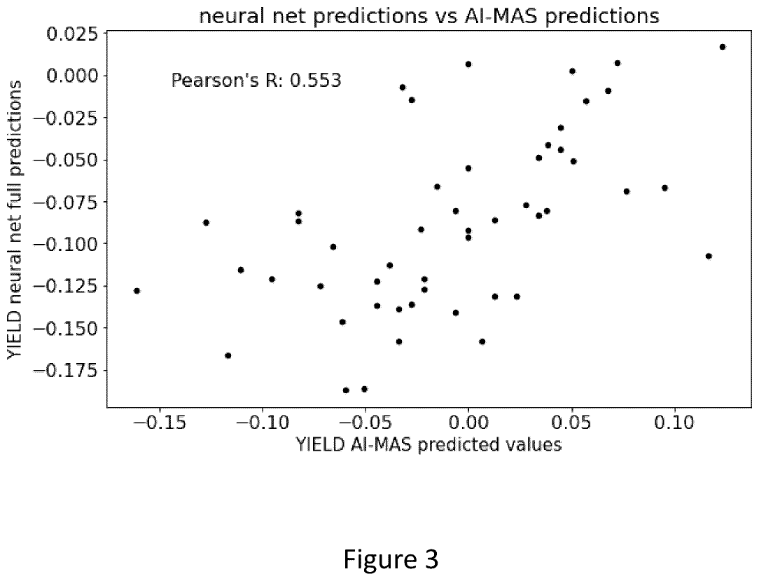

To estimate the potential value of AI-MAS at early stages of inbred line creation, F2 filial lines were simulated (as in Example 2), AI-MAS scores for those lines were generated, and simulated inbreeding was extended for several for generations to F4. AI-MAS predictions for F2 simulants were compared to the full neural net predictions from later simulated F4 generations (FIG. 3). This was performed separately for each trait of interest. This method allows for the assessment of the power of AI-MAS selections at early filial stages to improve breeding outcomes for later filial generations.

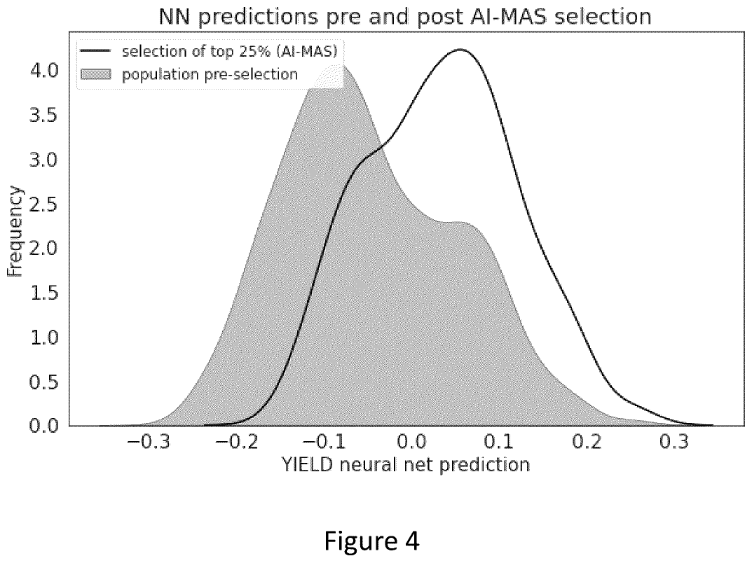

In detail, 50 filial lines were simulated from the observed genotypes of the parental lines. For each filial line, a Poisson distributed number of recombination events was selected uniformly along genetic space, individually for each chromosome. At simulated recombination sites the homozygous chromosomes of the parents inbred were exchanged, mimicking the biological phenomenon of genetic recombination. This process proceeds recursively, with additional rounds of recombination operating on the results of the previous round. This results in a reduction of heterozygosity and linkage disequilibrium in proportion to the number of filial generations simulated. In the first instance simulations are extend two generations in total, generating F₂ filial lines (although the number of generations is arbitrary). An estimate of the phenotypic value of F₂ simulated filial lines was generated using their genotypes at AI-MAS QTL (which are identified from simulated DHs), summing over allelic effects at those QTL using the standard procedure of AI-MAS. A selection regime to identify the F₂ lines with the highest probability of producing favorable F₄ lines was employed by ranking the F₂ by their AI-MAS predicted phenotypic scores and then selecting the top 25% of the lines (FIG. 4). Selection at the F₂ level was performed separately for each trait of interest.

For all F₂ lines additional generations of selfing were simulated, generally proceeding through to the fourth filial generation (although the final number of generations is arbitrary), mimicking the process of single seed descent, which is common in commercial breeding programs. For each filial line 25 F₄ lines were simulated. These extended simulations represent a typical terminal state at which new inbred lines would be selected for further advancement in a breeding program cycle. Using the full set of SNP markers neural nets are used to predict phenotype from the simulated genotype data. This allows for a good estimate of the simulated phenotypic value of each member of the derived F₄ generation.

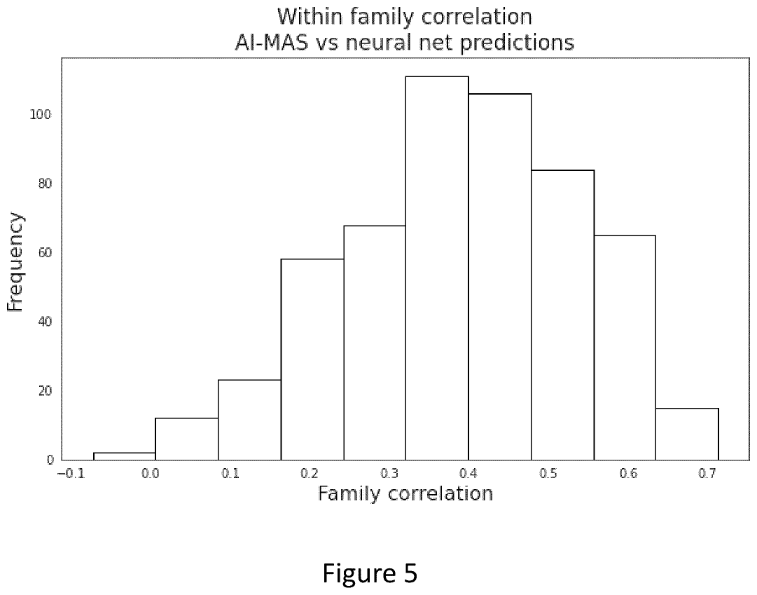

Having AI-MAS predicted phenotypes for F₂ lines, and whole genome predictions for their simulated F₄ lines allows the prioritization of families that are likely to respond well to AI-MAS. For example, the correlation of AI-MAS based phenotypic predictions and those derived from the neural net whole genome predictions were calculated, and families where the correlation were high were prioritized for AI-MAS (FIG. 5). This is because the marker restricted prediction of AI-MAS is a good approximation of a whole genome prediction. Furthermore, the estimated response to a selection regime employed at the F₂ level is assessed by comparing the distribution of predicted phenotypic values for the trait of interest in the original simulated F₄ population (i.e., the complete population of F₄ lines derived from all F₂ lines generated for a given family) with that of the reduced population of F₄ derived from the AI-MAS selected F₂ lines. A delta (δ) value is calculated for each trait and family as follows:

$$δ = {T}_{s} - {T}_{o}$$

where Tₛ is the mean phenotypic value of the selected population of F₄ lines, and Tₒ is the mean phenotypic value of the original population of F₄ lines. Delta (δ) values are normalized such that a positive value indicates movement towards to breeding target. Thus, a positive δ indicates a positive simulated outcome for AI-MAS for a given trait and family. The greater the response to selection, and the greater the correlation to full trait predictions, the greater the potential value of AI-MAS for a given family.

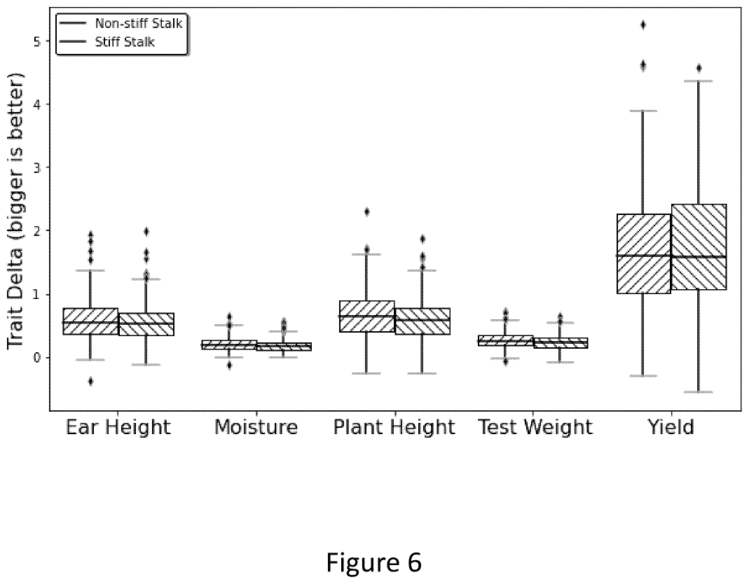

The Pearson's correlation coefficient between AI-MAS predictions from simulated F₂ material and full ANN based WGPs predictions for their derived F₄ offspring was: yield (mean=0.39, std=0.15), ear height (mean=0.42, std=0.14), moisture (mean=0.42, 0.14), plant height=0.38, std=0.15), test weight (mean=0.42, std=0.14). The mean delta (response to AI-MAS selection, described above) was: yield (mean=1.72, std=0.97), ear (height=0.57, std=0.31), moisture (mean=0.20, std=0.11), plant height (mean=0.63, std=0.35), test weight (mean=0.25, std=0.13) (FIG. 6).

Families where F₂ AI-MAS predictions were highly correlated with the full neural net predictions for their derived F₄s, or where delta values were particularly high, were identified and prioritized for future AI-MAS. Results for all 533 families analyzed suggests that almost all are likely to respond positively to AI-MAS selection and the utility of AI-MAS is likely broad across corn germplasm.

### Example 7

This example demonstrates the use of AI-MAS to predict genetic value in soybean using a Bayesian model.

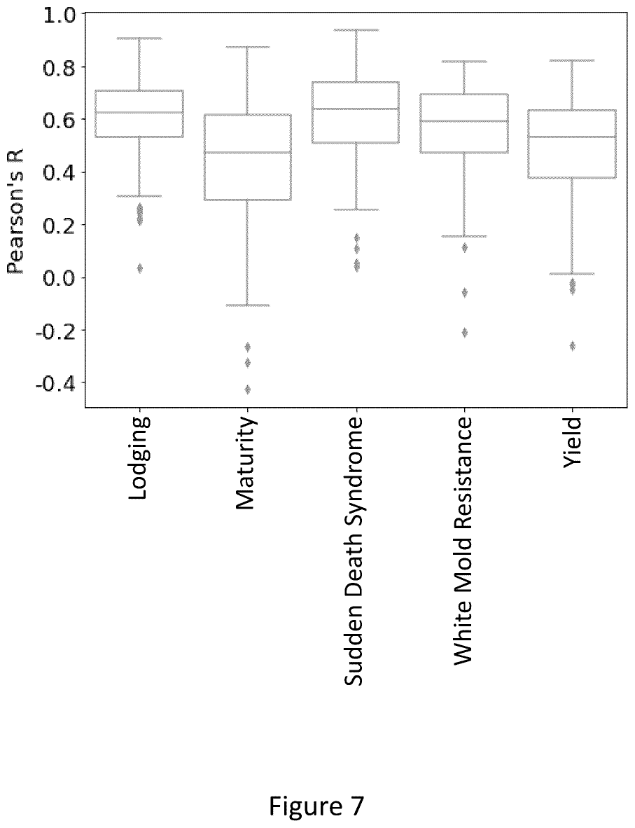

Simulated progeny populations from a soybean bi-parental cross were generated. Following the scheme outlined in Example 3, the accuracy of AI-MAS in soybean for generating AI-MAS GEBVs for the following traits: lodging, maturity, white mold resistance, sudden death syndrome resistance, and maturity adjusted yield and yield was assessed. In total 101 soybean families were included in the analysis and for each trait the within family correlation to traditional WGP values was calculated. The maturity of families included in the analysis ranged from 20 to 35. The AI-MAS GEBVs and traditional full WGP showed a mean Pearson's correlation of 0.53 (std=0.22). Stratified for each trait, correlations of WGP and AI-MAS GEBVs were as follows: yield (mean=0.48, std=0.22); maturity adjusted yield (mean=0.42, std=0.27); lodging (mean=0.60, std=0.17), maturity (mean=0.39, std=0.27); sudden death syndrome (mean=0.61, std=0.18) and white mold resistance (mean=0.55, std=0.19) (FIG. 7).

These results demonstrate the ability to predict SNP markers and phenotypes of simulated soybean populations using machine learning, and thus, allows obtaining data for mapping QTL loci and selecting few SNP markers predictive of performance.

### Example 8

This example demonstrates the use of AI-MAS to predict genetic value in soybean using an artificial neural network.

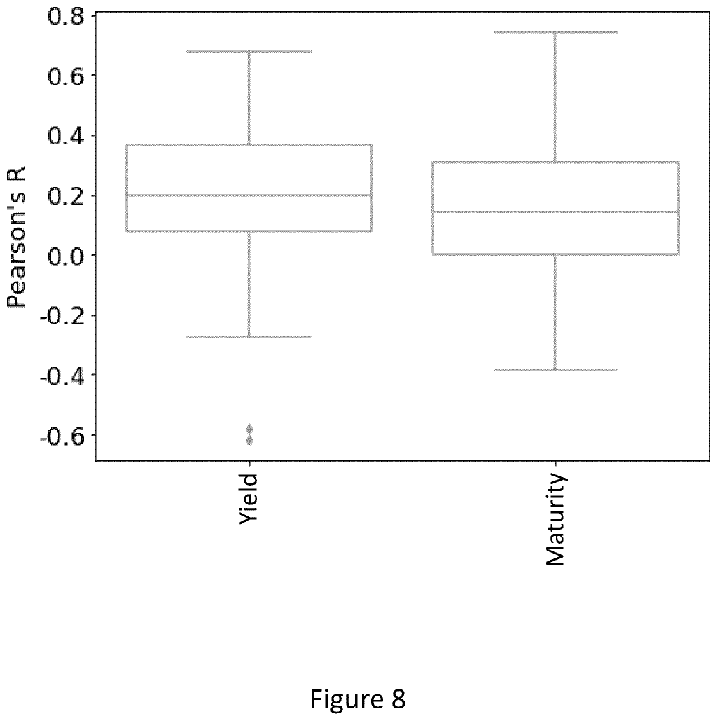

Simulated progeny populations from a soybean bi-parental cross were generated. Following the method outlined in Example 2, the accuracy of AI-MAS in soybean for generating AI-MAS GEBVs for maturity and yield was assessed. In total 128 soy families were included in the analysis and for each trait the within family correlation to traditional WGP values was calculated. The maturity of families included in the analysis ranged from 20 to 35. The AI-MAS GEBVs and traditional full WGP showed a mean Pearson's correlation of 0.18 (std=0.22). As shown in FIG. 8, stratified for each trait, correlations of WGP and AI-MAS GEBVs were as follows: yield (mean=0.21, std=0.24); maturity (mean=0.15, std=0.22).

These results demonstrate the ability to predict SNP markers and phenotypes of simulated soybean populations using machine learning, and thus, allows obtaining data for mapping QTL loci and selecting few SNP markers predictive of performance.

### Example 9

This example demonstrates that use of AI-MAS to select microspore embryos for breeding.

AI-MAS markers will be selected using simulated populations of a bi-parental breeding cross as described in Example 1. For a trait, or several traits, of interest the predicted phenotypes of the simulated population will be estimated as described in Example 2 and/or Example 3. Embryos will be non-destructively sampled for genetic material and genotyped at selected AI-MAS markers. Breeding value predictions (GEBVs) for microspore embryos will be calculated based on genotypes at AI-MAS markers following methods described herein.

Populations of embryos from a bi-parental breeding cross that are subjected to AI-MAS could range from 10, 20, 50, 100, 1000, 10,000, 100,000 or larger. Microspore embryo material can be selected for breeding purposes based on AI-MAS GEBVs, selecting the most favorable 10, 20, 30, 40, 50, 60, 70, 80 or 90 percent of material depending on the population or trait.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A method of selecting a member or members for a breeding program, the method comprising:
a. crossing at least two breeding partners in silico to create a simulated progeny population comprising a plurality of members;
b. inputting representations of genotypic information from the simulated progeny population into a trained a machine learning model to generate a predicted phenotypic profile for at least one trait of interest for one or more members of the plurality of members of the simulated progeny population, wherein the machine learning model has been trained to predict the at least one trait of interest;
c. identifying quantitative trait loci (QTL) associated with the at least one trait using the genotypic information and phenotypic profile of the plurality of members; and
d. identifying at least one allele of one or more polymorphic markers within or linked to the identified QTL, wherein the markers are polymorphic within the population.

2. The method of claim 1, wherein prior to (a) the method further comprises genotyping a sample from one or more of the at least two breeding partners.

3. The method of claim 1, wherein the representations of genotypic information for the population members are imputed or predicted from the at least two breeding partners.

4. The method of claim 1, wherein the simulated progeny population comprises a doubled haploid plant, an inbred plant, a hybrid plant, a microspore embryo, a haploid embryo, or an offspring derived from the member.

5. The method of claim 1, wherein the machine learning model is an artificial neural network (ANN).

6. The method of claim 1, wherein the machine learning model is trained to predict a trait selected from the group consisting of yield, plant height, seed moisture, predicted general combining ability (GCA), test weight, growing degree days for silking, ear height, brittle snap, early root lodging, late root lodging, or northern corn leaf blight, or any combination thereof.

7. The method of claim 1, wherein the QTLs associated with the at least one trait of interest are identified using compositive interval mapping.

8. The method of claim 1, wherein the identified alleles of the QTLs associated with the at least one trait of interest are assigned an allelic effect.

9. The method of claim 8, wherein the method further comprises ranking the additive effect of the at least one allele of the one or more polymorphic markers within or linked to the identified QTL.

10. The method of claim 1, wherein the method further comprises selecting a set of the one or more polymorphic markers that selectively identifies at least one member of the plurality of members having a desired genomic estimated breeding value (GEBV) for the at least one trait of interest.

11. The method of claim 10, wherein the set of the one or more polymorphic markers comprises the polymorphic marker having the largest additive effect for the at least one trait of interest.

12. The method of claim 10, wherein the number of polymorphic markers in the set of markers needed to identify a member with the desired GEBV for the at least one trait of interest is less than the number of polymorphic markers needed for genome prediction breeding.

13. The method of claim 1, wherein steps (b)-(d) are repeated at least one time for at least one additional trait of interest.

14. (canceled)

15. The method of claim 1, further comprising crossing the at least two breeding partners, genotyping the progeny, and selecting progeny comprising the at least one allele of one or more polymorphic markers within or linked to the identified QTL thereby selecting progeny having a desired GEBV for the least one trait of interest.

16. (canceled)

17. The method of claim 1, further comprising crossing the at least two breeding partners to produce a progeny population, detecting in the nucleic acid of a plant or embryo of the progeny population the at least one allele of one or more polymorphic markers within or linked to the identified QTL, selecting a plant or embryo comprising the at least one allele, crossing the selected plant or a plant generated from the selected embryo with a second plant to produce a population comprising the at least one allele of one or more polymorphic markers, and collecting the seeds produced thereby.

18. The method of claim 1, wherein the simulated progeny population is a microspore population.

19. The method of claim 18, further comprising crossing the at least two breeding partners to produce a progeny microspore population, detecting in the microspore population the at least one allele of one or more polymorphic markers within or linked to the identified QTL, selecting a microspore comprising the at least one allele, and generating a double haploid plant from the selected microspore.

20. The method of claim 19, further comprising crossing the generated double haploid plant with a second plant.

21. The method of claim 1, wherein step (d) comprises determining the LOD score for each of the identified polymorphic markers of a QTL.

22. The method of claim 21, wherein the method further comprises ranking the identified QTL based LOD score and selecting the identified QTL having the strongest linkage to the polymorphic markers for use in step (d).
